(19) United States
(12) Patent Application Publication
(10) Pub. No.: US 2018/0099114 A1
(43) Pub. Date: Apr. 12, 2018

(54) TACTILE STIMULATING DEVICE

(71) Applicant: Darryl Walski, Palos Heights, IL (US)

(72) Inventor: Darryl Walski, Palos Heights, IL (US)

(21) Appl. No.: 15/286,698

(22) Filed: Oct. 6, 2016

(51) Int. Cl. A61M 21/02; F04D 25/08; F04D 25/06; A61H 99/00

(52) U.S. Cl. CPC A61M 21/02; F04D 25/088; A61M 2021/0022; A61H 99/00; F04D 25/06

(57) ABSTRACT

A tactile stimulating device that is either floor mounted and portable, or a ceiling mounted unit that provides for therapeutic tactile stimulation to a person is disclosed. The tactile stimulating device rotates a variety of materials to create tactile stimulation in a random pattern on the user. The random stimulation pattern is achieved through rotation provided by a main rotational bar, followed by rotation of a first and second rotational bar that are located at opposite ends of the main rotational bar. The tactile stimulating device may be set up and operated by the user and has a timing device to shut down the device automatically, negating the need for the user to move to turn off the device after being in a relaxed state.

This invention relates to a tactile stimulating device and more particularly to a tactile stimulating device which has a main rotational bar, a first rotational bar, and a second rotational bar that provide random and chaotic motion and interchangeability of stimulating materials.

### BACKGROUND OF THE INVENTION

Tactile stimulation is known to be a relaxing and pleasant sensation. Tactile stimulation can aid in relaxation and sleep. However, tactile stimulation requires a person to physically touch another person which can be physically tiring and can detract from the privacy required for relaxation or sleep. A tactile stimulating device which can be positioned and operated independently by the user is a useful device.

A tactile stimulating device is convenient when it is actually being used. However, a tactile stimulating device can be bulky, heavy, and cumbersome when not in use. A tactile stimulating device may take up a great deal of physical space, may be bulky, heavy, and awkward to transport, or otherwise be obtrusive when it is not in use. A tactile stimulating device which is lightweight and can be folded down in a compact fashion for storage or transport is a useful device. Or, a tactile stimulating device that can be mounted directly to the ceiling structure of the building or a ceiling fan, thereby being ready for use, while remaining unobtrusive is a useful device.

Tactile stimulation can be pleasant and relaxing. Tactile stimulation which is random, chaotic, and unexpected can be even more pleasant and relaxing. A tactile stimulating device which provides for random and chaotic stimulation patterns is a useful device.

Each individual user has different preferences as to what is and is not pleasant and relaxing for tactile stimulation. A tactile stimulating device may use cloths or other materials to provide the tactile stimulation. While one user may prefer cloths, another user may prefer silk for optimal relaxation and sleep-inducement. A tactile stimulating device which can interchangeably account for different user preferences is a useful invention.

Tactile stimulation can relax a person and cause them to sleep. However, this relaxation and sleep-inducing purpose is defeated if a user must get up and physically turn off the tactile stimulating device once they are finished. A tactile stimulating device that can be set to shut off after a predetermined period of time without further user intervention is a useful device.

### SUMMARY OF THE INVENTION

A first objective of the present invention is the provision of a tactile stimulating device which can provide tactile stimulation without the need for another human to physically provide the tactile stimulation.

A second objective of the present invention is the provision of a tactile stimulating device which can provide tactile stimulation in the privacy of a bedroom or other suitable environment.

Another objective of the present invention is the provision of a tactile stimulating device which can be easily folded down into a compact position and be relatively unobtrusive and easy to transport when not in use.

Also, an objective of the present invention is the provision of a tactile stimulating device which can be mounted directly to the ceiling structure of a building or a ceiling fan, thereby being ready for use, while remaining unobtrusive.

A further objective of the present invention is the provision of a tactile stimulating device which can provide tactile stimulation in a random, chaotic, and unexpected fashion for optimal relaxation and sleep-inducing purposes.

A still further objective of the present invention is the provision of a tactile stimulating device which allows for an interchangeability of stimulating materials to account for individual user preferences.

Also, an objective of the present invention is the provision of a tactile stimulating device which can be set to operate for a predetermined period of time and automatically shut off after that period of time without further user intervention.

These and other objectives of the invention (which other objectives become clear by consideration of the specification, claims, and drawings as a whole) are met by providing a tactile stimulating device which has a main rotational bar, a first rotational bar, and a second rotational bar that provide random and chaotic motion and interchangeability of stimulating materials.

### BRIEF DESCRIPTION OF THE DRAWINGS

FIG. 1 depicts a right plan view of tactile stimulating device 100 mounted to base 120 with main conduit 126 partially depicted in phantom and with wire sets 230, 234, 236, and 242 fully depicted in phantom.

FIG. 2 depicts a top plan view of tactile stimulating device 100 mounted to base 120 with wire sets 230, 232, 234, and 236 fully depicted in phantom.

FIG. 3 depicts a rear plan view of tactile stimulating device 100 mounted to base 120 with main rotational motor wire set 230 depicted in phantom.

FIG. 4 depicts a top right perspective view of the junction of upright leg 122 and horizontal arm 124 at axle 162 with main rotational motor wire set 230, upright leg endplate 132, and axle 162 partially depicted in phantom and with horizontal arm end plate 130 fully depicted in phantom.

FIG. 5 depicts a rear perspective view of base 120 and upright leg mounting plate 160 in a partially exploded view.

FIG. 6 depicts a right plan view of tactile stimulating device 100 with arrows depicting movement to create folded position 110 with horizontal arm 124 partially depicted in phantom and horizontal arm 124, wire set 230, and main rotational shaft 142 fully depicted in phantom.

FIG. 7 depicts a rear plan view of tactile stimulating device 100 with horizontal arm 124 pivoted on axle 162 in the process of creating folded position 110 with main rotational motor wire set 230 fully depicted in phantom.

FIG. 8 depicts a top plan view of tactile stimulating device 100 in folded position 110 with main rotational motor wire set 230 fully depicted in phantom.

FIG. 9 depicts a top, right perspective view of main rotational bar 140, main rotational shaft 142, first rotational bar 144, first rotational shaft 146, second rotational bar 148, and second rotational shaft 150 with wire sets 234 and 236 depicted in phantom.

FIG. 10 depicts a bottom, left perspective view of electrical contact plate 204, positive ring 206, and negative ring 208.

FIG. 11 depicts a side plan view of tactile stimulating device 100 with ceiling mount 300 with wire sets 234, 236, and 242 depicted in phantom.

FIG. 12 depicts a side plan view of tactile stimulating device 100 with hard wire power source 310 in ceiling mount 300 with wire sets 234, 236, and 242 depicted in phantom.

FIG. 13 depicts a side plan view of tactile stimulating device 100 mounted in ceiling fan mount 400 and with overhead hard wire power source 410 and with wire sets 230, 234, 236, and 242 fully depicted in phantom.

Throughout the figures of the drawings, where the same part appears in one or more than one figure of the drawings, the same number is applied 110 thereto.

### DESCRIPTION OF THE PREFERRED EMBODIMENTS

Reference will now be made in detail to several embodiments of the invention that are illustrated in accompanying drawings. Whenever possible, the same or similar reference numerals are used in the drawings and the description to refer to the same or like parts or steps. The drawings are in simplified form and are not to precise scale. For purposes of convenience and clarity only, directional terms such as top, bottom, left, right, up, over, above, below, beneath, rear, and front, may be used with respect to the drawings. These and similar directional terms are not to be construed to limit the scope of the invention in any manner. The words attach, connect, couple, and similar terms with their inflectional morphemes do not necessarily denote direct or intermediate connections, but may also include connections through mediate elements or devices.

A tactile stimulating device is disclosed. In one embodiment, the tactile stimulating device has a base, an upright leg, a horizontal arm, a main rotational bar, a first rotational bar, a second rotational bar, and a series of motors and electrical connections. The tactile stimulating device has a folded position for easy storage and transportation when not in use. Or, in a second and third embodiment, the tactile stimulating device mounts directly to the ceiling structure of the building or to a ceiling fan, thereby being ready for use, while remaining unobtrusive.

The tactile stimulating device has a series of stimulating materials attached to a first rotational bar and a second rotational bar. A user usually lies on a bed or other surface and allows the stimulating materials to gently caress or brush upon the skin which provides a pleasant tickling, caressing, or other stimulation.

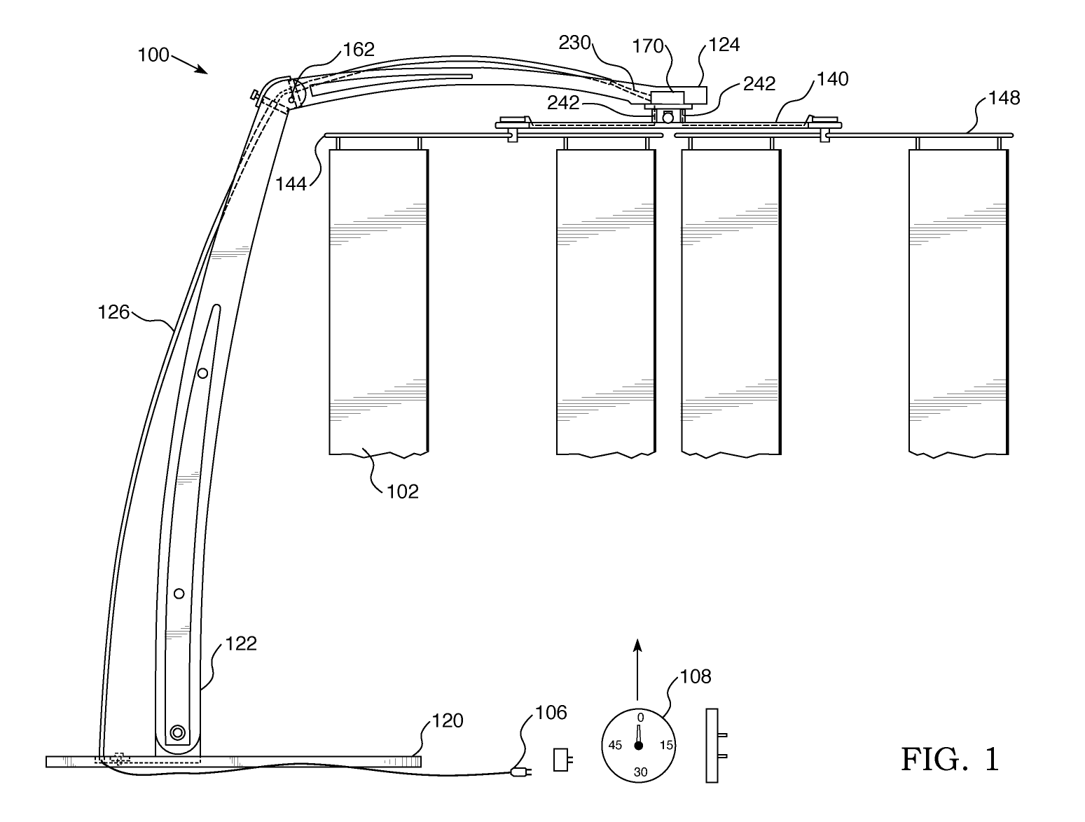

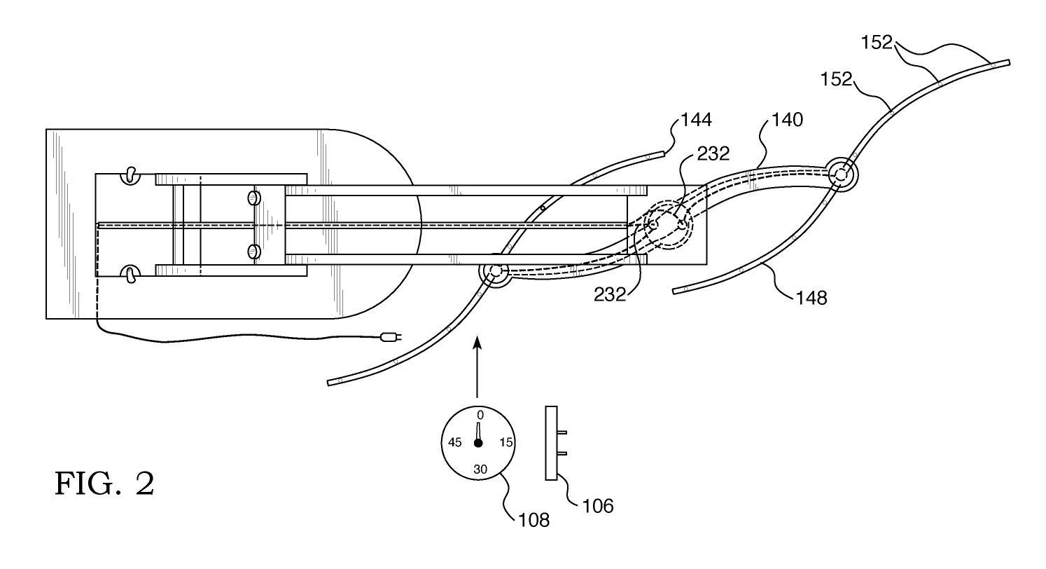

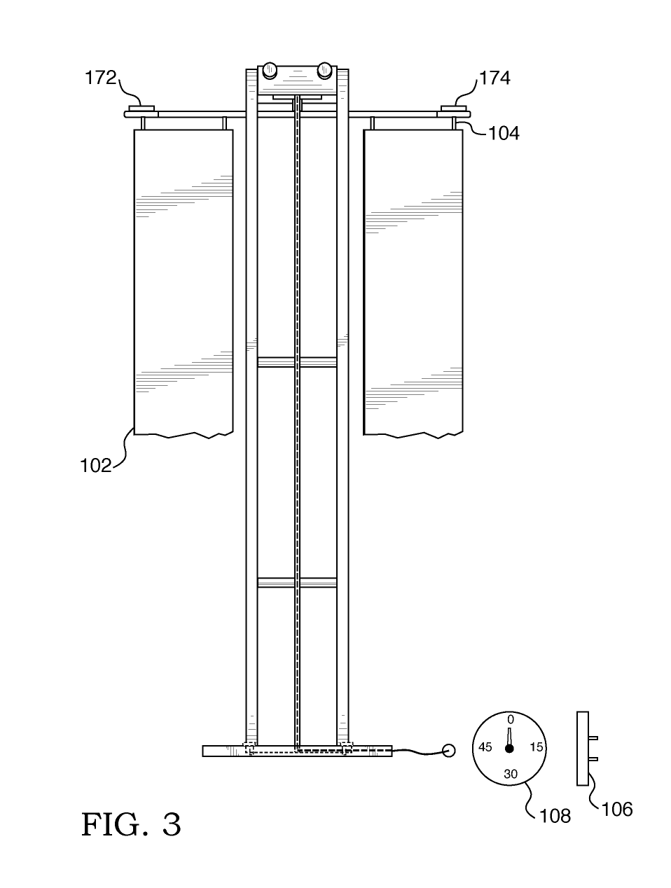

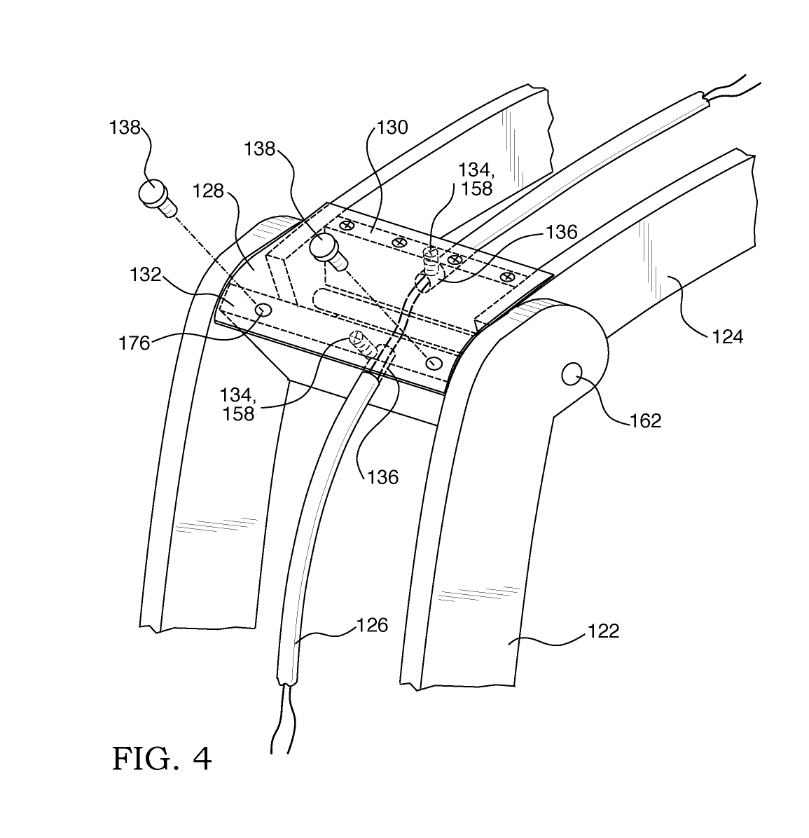

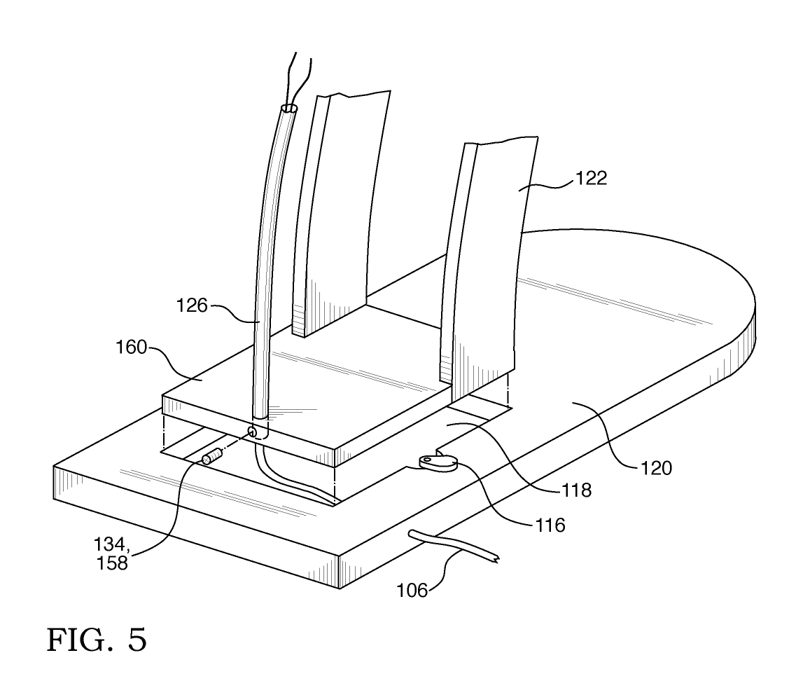

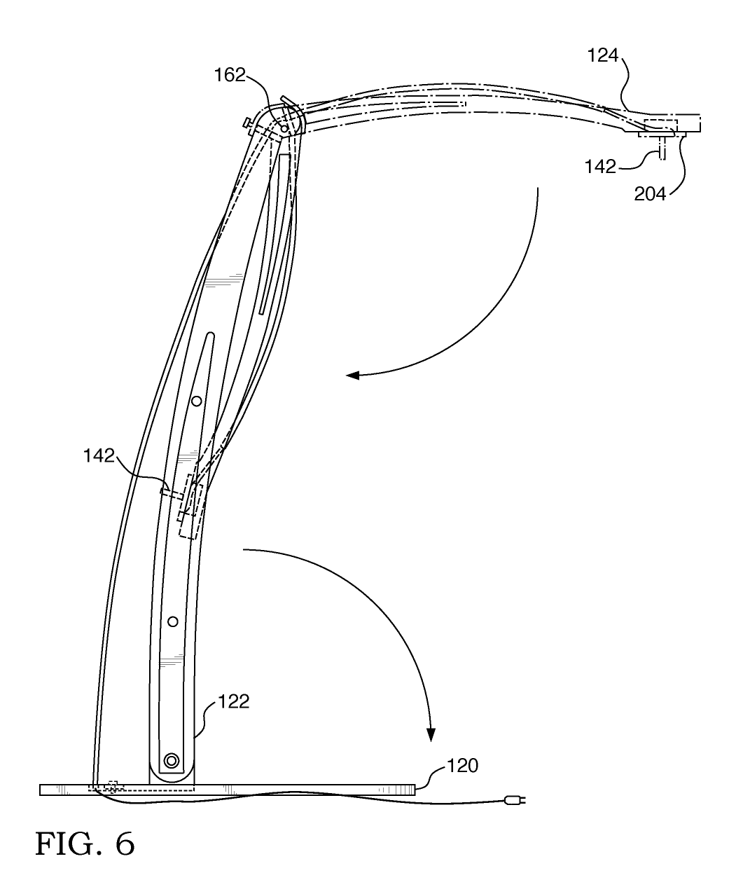

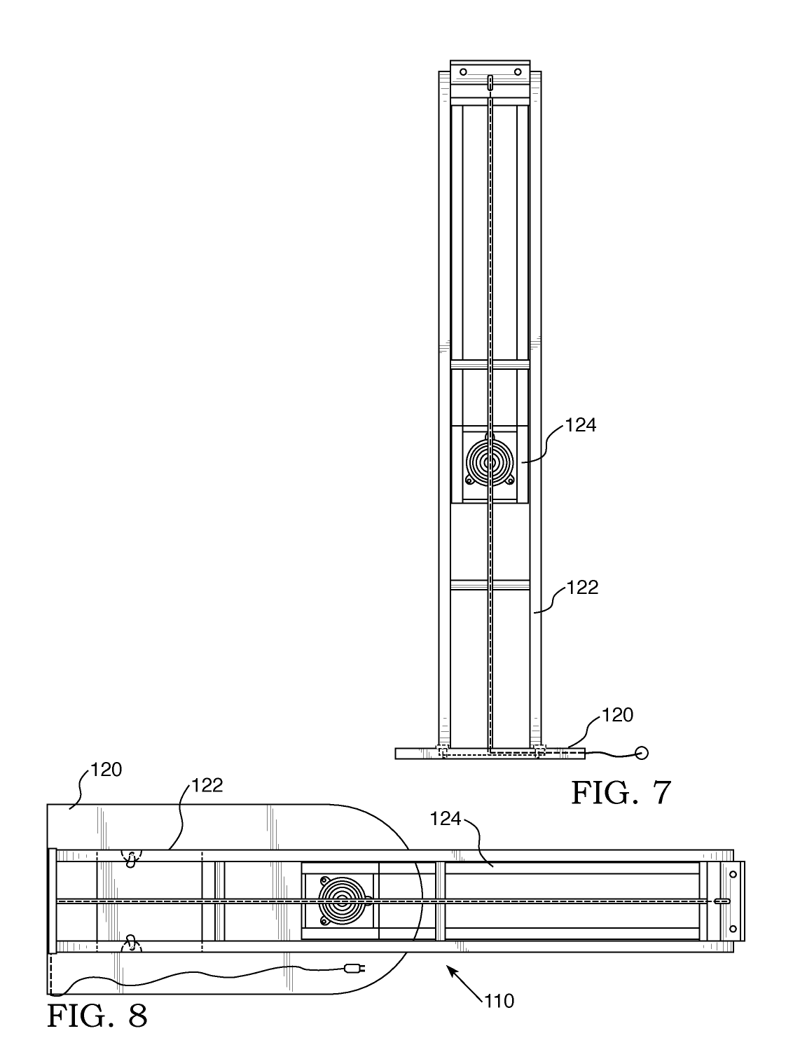

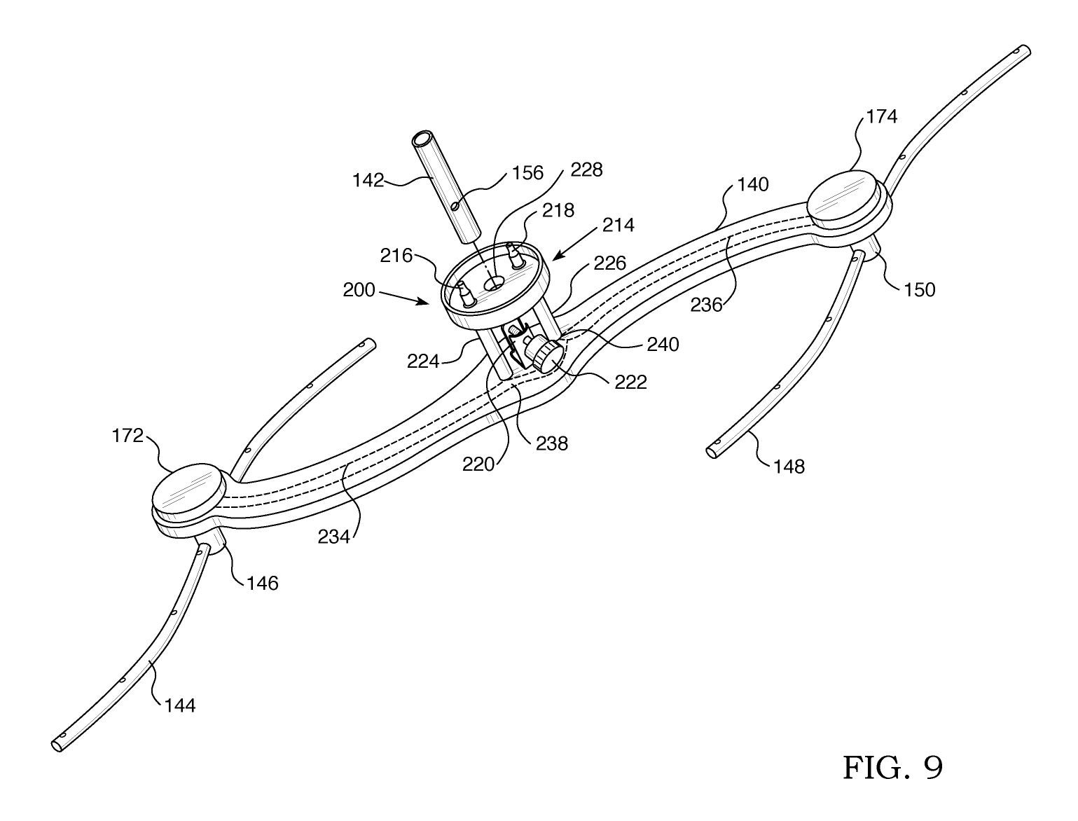

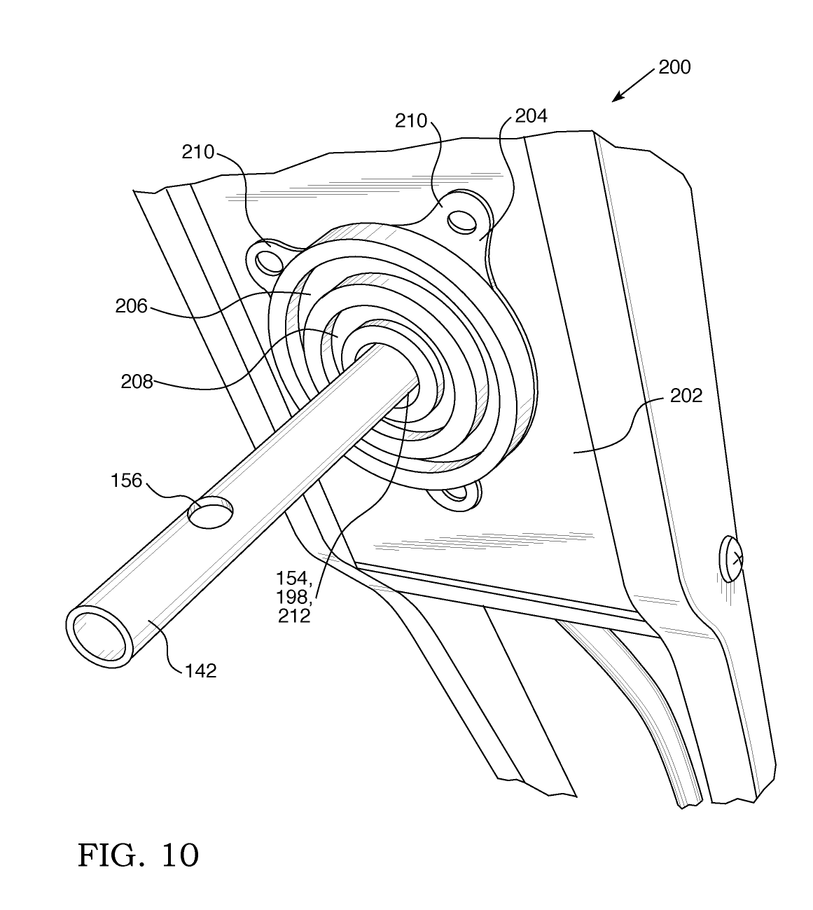

Now adding FIG. 1, FIG. 2, FIG. 3, FIG. 4, FIG. 5, FIG. 6, FIG. 7, FIG. 8, FIG. 9, and FIG. 10 to the consideration, the structure and function of tactile stimulating device 100 of this invention can be clearly seen. Tactile stimulating device 100 has base 120. Upright leg 122 is attached to and extends upwardly from base 120 via upright leg mounting plate 160. Horizontal arm 124 is pivotably attached to and extends horizontally from upright leg 122 via axle 162.

Main rotational motor 170 is attached within horizontal arm 124. Main rotational bar 140 is attached to main rotational motor 170 via main rotational shaft 142. Main rotational bar 140 has first rotational motor 172 and second rotational motor 174 on oppositely disposed ends. First rotational shaft 146 and second rotational shaft 150 extend from first rotational motor 172 and second rotational motor 174. First rotational bar 144 is attached to main rotational bar 140 through first rotational shaft 146 of first rotational motor 172. Second rotational bar 148 is attached to main rotational bar 140 through second rotational shaft 150 of second rotational motor 174.

First rotational bar 144 and second rotational bar 148 each have a series of mounting apertures 152. Mounting apertures 152 cooperate with fasteners 104 on stimulating materials 102 to removably secure stimulating materials 102 to first rotational bar 144 and second rotational bar 148. Fasteners 104 allow for stimulating materials 102 to be removed and replaced with other stimulating materials 02 to accommodate for different user preferences.

Fasteners 104 can be strings that are tied through the mounting apertures 152, hooks, wire hangers, or any other suitable fastening device that provides a secure, yet removable connection and all such fastening devices are encompassed by this disclosure.

Stimulating materials 102 can be cloth, silk, plastic, feathers, or any of a variety of stimulating materials that provide different levels of tactile stimulation and all such materials are encompassed by this disclosure.

Tactile stimulating device 100 has remote timer 108. Remote timer 108 allows for tactile stimulating device 100 to be turned on for a set period of time and then automatically stop. Remote timer 108 allows a user to turn on tactile stimulating device 100 and then go to bed without the need to later get out of bed to turn device 100 off.

Referring specifically to FIG. 4, FIG. 5, FIG. 6, FIG. 7, and FIG. 8, tactile stimulating device 100 has folded position 110 to provide for space saving storage and ease of transport when not in use. Folded position 110 is achieved by removing main rotational bar 140 from main rotational shaft 142. Main rotational bar 140 is secured to main rotational shaft 142 through the cooperation of knobbed attachment bolt 222 and knob aperture 156. Thus, by removing or loosening knobbed attachment bolt 222, main rotational bar 140 can be released from main rotational shaft 142.

Upright leg 122 has upright leg endplate 132 with center aperture 136. Horizontal arm 124 has horizontal arm endplate 130 with center aperture 136. A first section of main conduit 126 travels from upright leg mounting plate 160 to upright leg endplate 132 and inserts into center aperture 136. A second piece of main conduit 126 travels from main rotational motor 170 to horizontal arm endplate 130 and inserts into center aperture 136. Main conduit attachment bolt 158 inserts into main conduit bolt aperture 134 on each of the horizontal arm endplate 130 and upright leg endplate 132 and secures the first section and the second section of main conduit 126 in center aperture 136 through friction.

Joining plate 128 is attached to horizontal arm 124. Horizontal arm 124 pivots on axle 162 to a horizontal position. Joining plate 128 extends over the junction of upright leg 122 and horizontal arm 124 and is secured in place through the cooperation of joining plate bolts 138 and joining plate apertures 176 on upright leg 122. To create the folded position 110, the joining plate bolts 138 are released and horizontal arm 124 pivots to a downward position on axle 162 through gravity.

Upright leg 122 and main conduit 126 are attached to upright leg mounting plate 160. Base 120 has mounting plate cavity 118 to receive upright leg mounting plate 160. Upright leg mounting plate 160 is secured in mounting plate cavity 118 through latches 116.

To create the folded position, upright leg mounting plate 160 is removed from mounting plate cavity 118 and thus, separated from base 120. Horizontal arm 124 is resting against upright leg 122 in folded position 110.

This creates folded position 110 which provides a compact form for tactile stimulating device 100 that can be easily stored or transported.

Referring specifically to FIG. 2, FIG. 9, and FIG. 10, tactile stimulating device 100 has main rotational bar 140 rotating at a predetermined amount of revolutions per minute (“RPM”). First rotational bar 144 is attached to main rotational bar 140 and rotates at a predetermined RPM. Second rotational bar 148 is attached to main rotational bar 140 and rotates at a predetermined RPM. The predetermined RPM of main rotational bar 140 can be the same or different from the predetermined RPM of first rotational bar 144 and second rotational bar 148.

First rotational bar 144 and second rotational bar 148 can rotate in the same or opposite directions of each other. In either case, the rotation of the main rotational bar 140 and the secondary rotation of first rotational bar 144 and second rotational bar 148 creates the random and chaotic tactile stimulation.

For first rotational bar 144 and second rotational bar 148 to rotate in the same direction, first rotational motor 172 and second rotational motor 174 must have their wire windings in the same direction. For first rotational bar 144 and second rotational bar 148 to rotate in opposite directions of each other, second rotational motor 174 must have its wire windings in an opposite direction of that of first rotational motor 172.

Now referring specifically to FIG. 9 and FIG. 10, the structure and function of rotational electrical connection 200 can be clearly seen. Motor mounting plate 202 is attached to the bottom of horizontal arm 124 and just beneath the main rotational motor 170. Electrical contact plate 204 is attached to the bottom of motor mounting plate 202. In the depicted embodiment, electrical contact plate 204 is glued to motor mounting plate 202. However, electrical contact plate 204 has mounting tabs 210 to accommodate screws or other suitable fasteners and all such suitable fasteners are encompassed by this disclosure.

Male mounting assembly 214 is attached to the top side of main rotational bar 140 through male mounting assembly bracket 220. Male mounting assembly bracket 220 can be attached to main rotational bar 140 through screws or any other suitable fasteners and all such suitable fasteners are encompassed by this disclosure.

Male mounting assembly 214 has positive wire conduit 224 and negative wire conduit 226 attached thereto. Male mounting assembly 214 also has spring loaded positive connector 216 and spring loaded negative connector 218. Male mounting assembly bracket 220 has knobbed attachment bolt 222 releaseably attached thereto.

Main rotational motor 170 is connected to power supply 106 through main rotational motor wire set 230. Main rotational motor wire set 230 starts at power supply 106 as alternating current (hereinafter “AC”) and travels through main conduit 126, through horizontal arm 124, then to main rotational motor 170 to provide for rotation of the main rotational shaft 142.

Electrical contact plate wire set 232 pigtails from main rotational motor wire set 230 and is electrically connected to rotational electrical connection 200. Electrical contact plate 204 has positive ring 206 and negative ring 208. Electrical contact plate wire set 232 ends at positive ring 206 and negative ring 208. Positive ring 206 and negative ring 208 are made of copper.

Positive and negative connector wire set 242 starts at spring loaded positive connector 216 and spring loaded negative connector 218 and travels through positive wire conduit 224 and negative wire conduit 226. Positive wire of set 242 travels from spring loaded positive connector 216, through positive wire conduit 224, and bifurcates at positive bifurcation 238. Then, positive wire of set 242 becomes part of first rotational motor wire set 234 and second rotational motor wire set 236. Negative wire of set 242 travels from spring loaded negative connector 218, through negative wire conduit 226, and bifurcates at negative bifurcation 240. Then, negative wire of set 242 becomes part of first rotational motor wire set 234 and second rotational motor wire set 236.

First rotational motor wire set 234 starts at positive bifurcation 238 and negative bifurcation 240 within main rotational bar 140. Then, first rotational motor wire set 234 travels to first rotational motor 172 to provide for rotation of the first rotational shaft 146.

Second rotational motor wire set 236 starts at positive bifurcation 238 and negative bifurcation 240 within the main rotational bar 140. Then, second rotational motor wire set 236 travels to second rotational motor 174 to provide for rotation of the second rotational shaft 150.

Horizontal arm 124 has main rotational shaft aperture 154. Motor mounting plate 202 has motor mounting plate aperture 198. Electrical contact plate 204 has contact plate aperture 212. Main rotational shaft 142 has knob aperture 156. Male mounting assembly 214 has mounting assembly aperture 228. Main rotational motor 170 is attached within horizontal arm 124. Main rotational shaft 142 inserts through main rotational shaft aperture 154, through motor mounting plate aperture 198, through contact plate aperture 212, through mounting assembly aperture 228, and the connection is secured through the cooperation of knobbed attachment bolt 222 which threads through male mounting assembly bracket 220, passing through knob aperture 156.

Spring loaded positive connector 216 and spring loaded negative connector 218 interact with positive ring 206 and negative ring 208 to maintain a constant electrical connection to provide power to first rotational motor 172 and second rotational motor 174. Spring loaded positive connector 216 and spring loaded negative connector 218 are part of male mounting assembly 214 and thus, are attached to main rotational bar 140 and rotate with it. Positive ring 206 and negative ring 208 are attached to electrical contact plate 204 and remain stationary relative to electrical contact plate 204. As spring loaded positive connector 216 and spring loaded negative connector 218 rotate within positive ring 206 and negative ring 208, a continuous flow of AC current is delivered to positive and negative connector wire sets 242 and subsequently to first rotational motor wire set 234 and second rotational motor wire set 236.

Spring loaded positive connector 216 and spring loaded negative connector 218 are spring loaded to cause them to stay in constant contact with positive ring 206 and negative ring 208 and to allow for rotational movement upon the positive ring 206 and negative ring 208. Connectors 216 and 218 will wear over time and spring loading them will allow them to stay in constant contact with positive ring 206 and negative ring 208 as they wear.

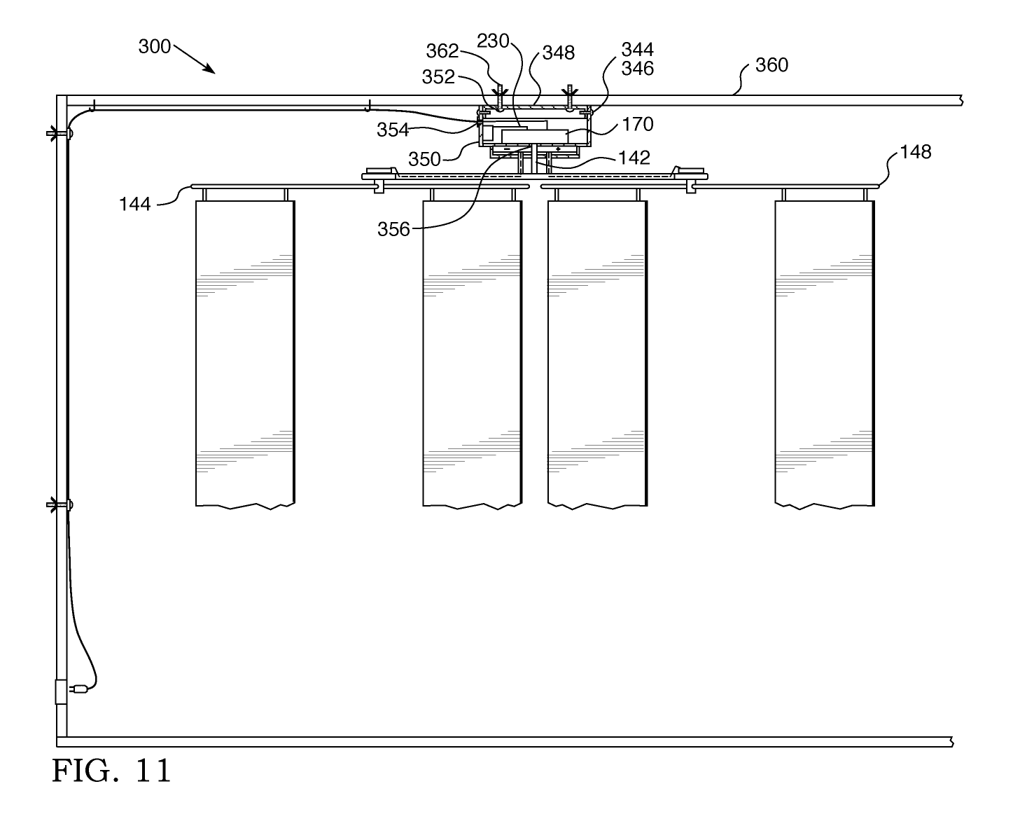

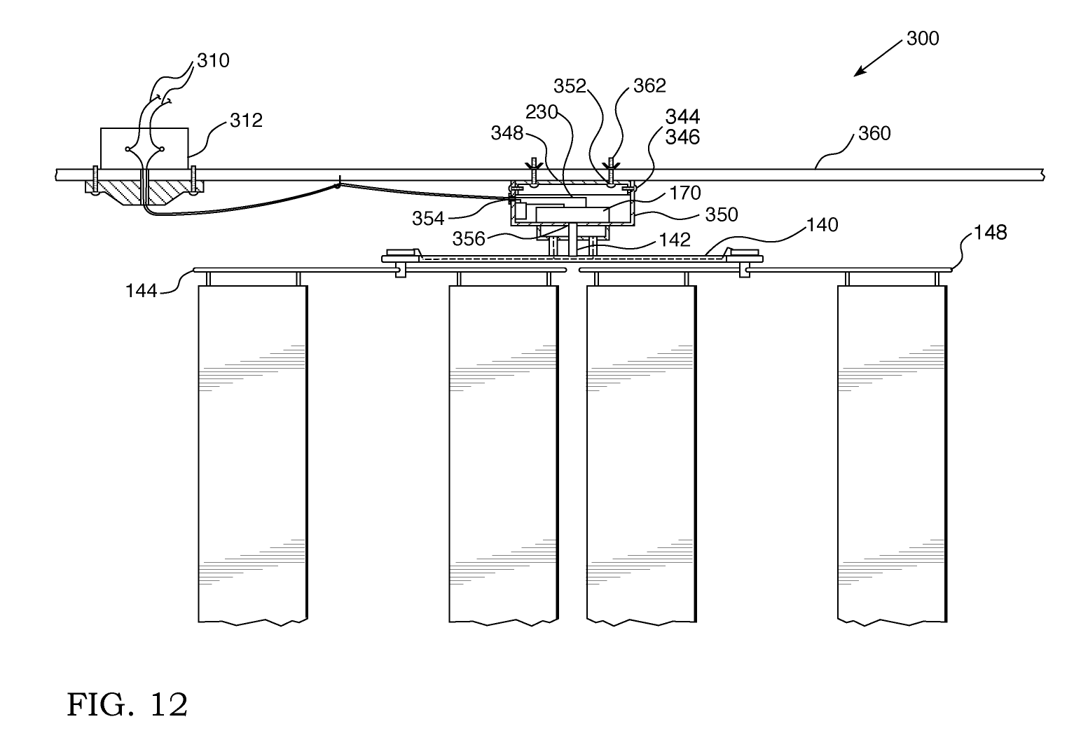

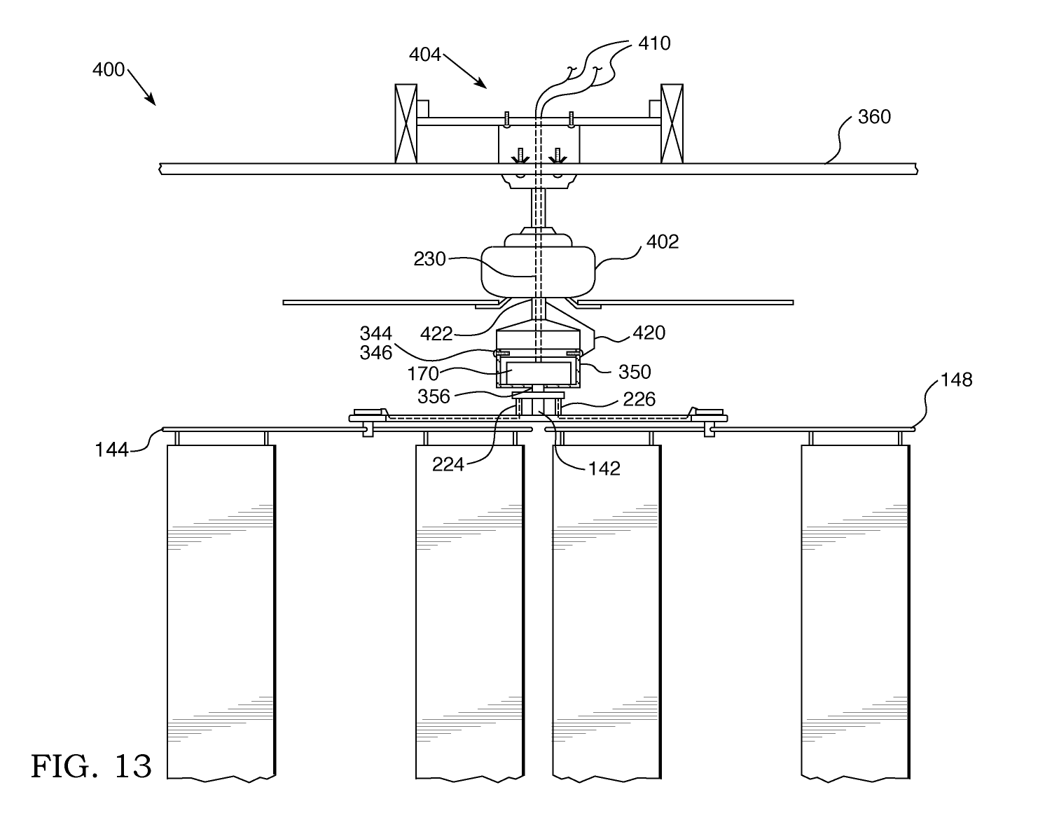

Now adding FIG. 11, FIG. 12, and FIG. 13 to the consideration, the structure and function of alternative embodiments of tactile stimulating device 100 can be clearly seen. Tactile stimulating device 100 can be mounted to a truss 360 of the ceiling of the building or can be mounted through a light adapter kit 420 of ceiling fan 402.

Now referring specifically to FIG. 11, ceiling mount 300 of tactile stimulating device 100 can be clearly seen. In this embodiment, main rotational motor housing 350 cooperates with mounting plate 348 and truss 360 within the house or building's ceiling structure to create ceiling mount 300.

Main rotational motor housing 350 encases main rotational motor 170. Main rotational motor housing 350 attaches to mounting plate 348 at junction apertures 346 and the connection is secured through fasteners 344. On the exterior bottom of main rotational motor housing 350 is motor mounting plate 202 and electrical contact plate 204.

Mounting plate 348 has mounting apertures 352 which cooperate with fasteners 362 to secure mounting plate 348 to truss 360. Main rotational motor housing 350 also has at least one power cord aperture 354 to allow a power cord 106 to extend from the outlet or power source 106 to power cord aperture 354. Furthermore, main rotational motor housing 350 has a main rotational shaft aperture 356 to allow the main rotational shaft 142 of main rotational motor 170 to attach to main rotational bar 140 as seen in FIG. 9 and FIG. 10.

Main rotational motor wire set 230 extends from power cord 106 of power source 106. Main rotational motor wire set 230 starts at power cord 106 and terminates at main rotational motor 170. The remainder of the wiring sets 232, 234, 236, and 242 for this embodiment are the same as described above in FIG. 9 and FIG. 10.

In the preferred embodiment, there are two pairs of apertures 346 and 352 and two pairs of fasteners 344 and 362. However, any suitable number of apertures 346 and 352 and fasteners 344 and 362 can be utilized to secure mounting plate 348 to truss 360 and main rotational motor housing 350 to mounting plate 348. Also, in the preferred embodiment, fasteners 344 and 362 are screws and carriage bolts, but any suitable fastener such as lag screws or molly bolts can be utilized and are encompassed by this disclosure.

Now referring specifically to FIG. 12, another embodiment of ceiling mount 300 of tactile stimulating device 100 can be clearly seen. In FIG. 12, a hard wire power source 310 utilizing a ceiling light junction box 312 is shown. Otherwise, ceiling mount 300 is identical to that depicted in FIG. 11.

Main rotational motor 170 is powered by hard wire power source 310 through power cord 106. Hard wire power source 310 is designed to power a standard ceiling light fixture. In this embodiment, the hard wire power source 310 for the standard ceiling light is instead wired to main rotational motor 170 through power cord 106, and bifurcates to provide power to both first rotational motor 172 and second rotational motor 174 through rotational electrical connection 200 as seen in FIG. 9 and FIG. 10.

Main rotational motor wire set 230 extends from power cord 106 of power source 106. Main rotational motor wire set 230 starts at power cord 106 and terminates at main rotational motor 170. The remainder of the wiring sets 232, 234, 236, and 242 for this embodiment is the same as described above in FIG. 9 and FIG. 10.

Now referring specifically to FIG. 13, ceiling fan mount 400 of tactile stimulating device 100 is shown. In this embodiment, tactile stimulating device 100 is hard wired into the ceiling fan 402 through a light adapter kit 420.

Ceiling fan 402 is attached to truss 360 through ceiling fan mount with integral junction box 404. Ceiling fan mount within integral junction box 404 attaches to ceiling fan 402 in an industry standard fashion. Many ceiling fan mounts with integral junction boxes 404 exist and are commercially available with many makes and models of ceiling fan 402. All such ceiling fan mounts with integral junction boxes 404 are encompassed by this disclosure.

Light adapter kit 420 attaches to ceiling fan 402 at light adapter mounting point 422 at the bottom of ceiling fan 402. Light adapter kits 420 exist and are commercially available with many makes and models of ceiling fan 402. All such light adapter kits 420 are encompassed by this disclosure.

One example of a commercially available light adapter kit 420 is available through Hunter Fan Company as Hunter 25264 Globe Adapter Kit in Weathered Brick. Hunter is a registered trademark, Registration Number 1,403,033 (among many), which is owned by Hunter Fan Company, a Corporation organized under the laws of the State of Tennessee, 2500 Frisco Avenue, Memphis, Tenn., 38114.

Main rotational motor housing 350 encases main rotational motor 170. Main rotational motor housing 350 attaches to light adapter kit 420 at junction apertures 346 and the connection is secured through fasteners 344. On the exterior bottom of main rotational motor housing 350 is motor mounting plate 202 and electrical contact plate 204.

Furthermore, main rotational motor housing 350 has main rotational shaft aperture 356 to allow the main rotational shaft 142 of main rotational motor 170 to attach to main rotational bar 140 as seen in FIG. 9 and FIG. 10.

Main rotational motor 170 is powered by overhead hard wire power source 410. Overhead hard wire power source 410 is designed to power ceiling fan 402 as well as an optional light accessory. Overhead hard wire power source 410 is wired within ceiling fan mount with integral junction box 404 to provide power for main rotational motor wire set 230. In this embodiment, the overhead hard wire power source 410 for the light accessory is instead wired to main rotational motor 170, and bifurcates to provide power to both first rotational motor 172 and second rotational motor 174 through rotational electrical connection 200 as seen in FIG. 9 and FIG. 10.

Main rotational motor wire set 230 begins at overhead hard wire power source 410 and terminates at main rotational motor 170. Ceiling fan 402, light adapter kit 420, and main rotational motor housing 350 act as a conduit for main rotational motor wire set 230. The remainder of the wiring sets 232, 234, 236, and 242 for this embodiment are the same as described above in FIG. 9 and FIG. 10.

This application—taken as a whole with the abstract, specification, claims, and drawings—provides sufficient information for a person having ordinary skill in the art to practice the invention disclosed and claimed herein.

Any measures necessary to practice this invention are well within the skill of a person having ordinary skill in this art after that person has made a careful study of this disclosure.

Because of this disclosure and solely because of this disclosure, modification of this apparatus and method can become clear to a person having ordinary skill in this particular art. Such modifications are clearly covered by this disclosure.

What is claimed and sought to be protected by Letters Patent is:

1. A tactile stimulating device for providing tactile stimulation comprising:
a) the tactile stimulating device having a base with an upright leg extending upwardly therefrom;
b) a horizontal arm extending horizontally from the upright leg;
c) a main rotational bar being attached to the horizontal arm;
d) at least one secondary rotational bar being attached to the main rotational bar;
e) at least one stimulating material being attached to the at least one secondary rotational bar; and
f) at least one motor providing power for the tactile stimulating device.

2. The tactile stimulating device of claim 1 further comprising:
a) the at least one rotational motor being a main rotational motor, a first rotational motor, and a second rotational motor;
b) the at least one secondary rotational bar connected to the main rotational bar being a first rotational bar and a second rotational bar; and
c) the first rotational bar and the second rotational bar being oppositely disposed on the main rotational bar.

3. The tactile stimulating device of claim 2 further comprising:
a) the main rotational motor being connected to the main rotational bar through a main rotational shaft;
b) the first rotational motor being connected to the first rotational bar through a first rotational shaft;
c) the second rotational motor being connected to the second rotational bar through a second rotational shaft;
d) a rotational electrical connection providing power to the first rotational motor and the second rotational motor; and
e) the rotational electrical connection having an electrical contact plate and a male mounting assembly.

4. The tactile stimulating device of claim 3 further comprising:
a) the electrical contact plate having a positive ring and a negative ring; and
b) the male mounting assembly having a spring loaded positive connector and a spring loaded negative connector.

5. The tactile stimulating device of claim 4 further comprising:
a) the horizontal arm having a motor mounting plate attached to an end thereof and below the main rotational motor;
b) a main conduit for housing a main rotational motor wire set with both the main conduit and the main rotational motor wire set extending from the base to the main rotational motor and with the main rotational motor wire set connecting to the power supply;
c) an electrical contact plate wire set extending from the main rotational motor wire set to the electrical contact plate and terminating at the positive ring and the negative ring;
d) a positive and negative connector wire set wherein, a positive wire from the set extends from the spring loaded positive connector, passes through a positive wire set conduit, and bifurcates at a positive bifurcation to a first rotational motor wire set and a second rotational motor wire set and further wherein, a negative wire from the set extends from the spring loaded negative connector, passes through a negative wire set conduit, and bifurcates at a negative bifurcation to the first rotational motor wire set and the second rotational motor wire set;
e) the first rotational motor wire set extending from the positive bifurcation and the negative bifurcation, passing through the main rotational bar, and terminating at the first rotational motor; and
f) the second rotational motor wire set extending from the positive bifurcation and the negative bifurcation, passing through the main rotational bar, and terminating at the second rotational motor.

6. The tactile stimulating device of claim 5 further comprising:
a) the horizontal arm having a main rotational shaft aperture;
b) the motor mounting plate having a motor mounting plate aperture;
c) the electrical contact plate having a contact plate aperture;
d) the male mounting assembly having a mounting assembly aperture;
e) the main rotational shaft having a knob aperture;
f) the main rotational shaft cooperating with the main rotational shaft aperture, the motor mounting plate aperture, the contact plate aperture, and the mounting assembly aperture; and
g) a knobbed attachment bolt cooperating with the knob aperture to secure the connection of the main rotational shaft in the main rotational shaft aperture, the motor mounting plate aperture, the contact plate aperture, and the mounting assembly aperture.

7. The tactile stimulating device of claim 6 further comprising:
a) at least one upright leg mounting plate connecting the upright leg to the base;
b) at least one axle connecting the horizontal arm to the upright leg;
c) the tactile stimulating device having a remote timer; and
d) the tactile stimulating device having a folded position.

8. A tactile stimulating device for providing tactile stimulation comprising:
a) the tactile stimulating device being attached to a ceiling fan through a light adapter kit;
b) at least one motor providing power for the tactile stimulating device;
c) a main rotational bar being attached to the at least one motor;
d) at least one secondary rotational bar being attached to the main rotational bar; and
e) at least one stimulating material being attached to the at least one secondary rotational bar.

9. The tactile stimulating device of claim 8 further comprising;
a) the tactile stimulating device having a main rotational motor housing wherein, the main rotational motor housing attaches to the light adapter kit and the light adapter kit mounts to the ceiling fan at a mounting point and extends below the ceiling fan;
b) the tactile stimulating device having a hard wire power source to a ceiling fan junction box;
c) the at least one rotational motor being a main rotational motor, a first rotational motor, and a second rotational motor;
d) the at least one secondary rotational bar connected to the main rotational bar being a first rotational bar and a second rotational bar; and
e) the first rotational bar and the second rotational bar being oppositely disposed on the main rotational bar.

10. The tactile stimulating device of claim 9 further comprising:
a) the main rotational motor being connected to the main rotational bar through a main rotational shaft;
b) the first rotational motor being connected to the first rotational bar through a first rotational shaft; and
c) the second rotational motor being connected to the second rotational bar through a second rotational shaft.

11. The tactile stimulating device of claim 10 further comprising:
a) a rotational electrical connection providing power to the first rotational motor and the second rotational motor;
b) the rotational electrical connection having an electrical contact plate and a male mounting assembly;
c) the electrical contact plate having a positive ring and a negative ring; and
d) the male mounting assembly having a spring loaded positive connector and a spring loaded negative connector.

12. The tactile stimulating device of claim 11 further comprising:
a) the main rotational motor housing having a motor mounting plate attached to an end thereof;
b) the ceiling fan and the light adapter kit acting as a conduit for housing a main rotational motor wire set, with the main rotational motor wire set extending from an overhead hard wire power source to the main rotational motor;
c) an electrical contact plate wire set extending from the main rotational motor wire set to the electrical contact plate and terminating at the positive ring and the negative ring;
d) a positive and negative connector wire set wherein, a positive wire from the set extends from the spring loaded positive connector, passes through a positive wire set conduit, and bifurcates at a positive bifurcation to a first rotational motor wire set and a second rotational motor wire set and further wherein, a negative wire from the set extends from the spring loaded negative connector, passes through a negative wire set conduit, and bifurcates at a negative bifurcation to the first rotational motor wire set and the second rotational motor wire set;
e) the first rotational motor wire set extending from the positive bifurcation and the negative bifurcation, passing through the main rotational bar, and terminating at the first rotational motor; and
f) the second rotational motor wire set extending from the positive bifurcation and the negative bifurcation, passing through the main rotational bar, and terminating at the second rotational motor.

13. The tactile stimulating device of claim 12 further comprising:
a) the main rotational motor housing having a main rotational shaft aperture;
b) the motor mounting plate having a motor mounting plate aperture;
c) the electrical contact plate having a contact plate aperture;
d) the male mounting assembly having a mounting assembly aperture;
e) the main rotational shaft having a knob aperture;
f) the main rotational shaft cooperating with the main rotational shaft aperture, the motor mounting plate aperture, the contact plate aperture, and the mounting assembly aperture; and
g) a knobbed attachment bolt cooperating with the knob aperture to secure the connection of the main rotational shaft in the main rotational shaft aperture, the motor mounting plate aperture, the contact plate aperture, and the mounting assembly aperture.

14. A tactile stimulating device for providing tactile stimulation comprising:
a) the tactile stimulating device being attached to a truss of a building;
b) at least one motor providing power for the tactile stimulating device;
c) a main rotational bar being attached to the at least one motor;
d) at least one secondary rotational bar being attached to the main rotational bar; and
e) at least one stimulating material being attached to the at least one secondary rotational bar.

15. The tactile stimulating device of claim 14 further comprising;
a) the tactile stimulating device having a main rotational motor housing wherein, the main rotational motor housing attaches to a mounting plate and the mounting plate is secured to the truss of the building;
b) the at least one rotational motor being a main rotational motor, a first rotational motor, and a second rotational motor;
c) the at least one secondary rotational bar connected to the main rotational bar being a first rotational bar and a second rotational bar; and
d) the first rotational bar and the second rotational bar being oppositely disposed on the main rotational bar.

16. The tactile stimulating device of claim 1 5 further comprising:
a) the main rotational motor being connected to the main rotational bar through a main rotational shaft;
b) the first rotational motor being connected to the first rotational bar through a first rotational shaft;
c) the second rotational motor being connected to the second rotational bar through a second rotational shaft;
d) a rotational electrical connection providing power to the first rotational motor and the second rotational motor;
e) the rotational electrical connection having an electrical contact plate and a male mounting assembly;
f) the electrical contact plate having a positive ring and a negative ring; and
g) the male mounting assembly having a spring loaded positive connector and a spring loaded negative connector.

17. The tactile stimulating device of claim 16 further comprising:
a) the main rotational motor housing having a motor mounting plate attached to an end thereof;
b) a main rotational motor wire set extending from a power cord to the main rotational motor and with the power cord connecting to the power supply;
c) an electrical contact plate wire set extending from the main rotational motor wire set to the electrical contact plate and terminating at the positive ring and the negative ring;
d) a positive and negative connector wire set wherein, a positive wire from the set extends from the spring loaded positive connector, passes through a positive wire set conduit, and bifurcates at a positive bifurcation to a first rotational motor wire set and a second rotational motor wire set and further wherein, a negative wire from the set extends from the spring loaded negative connector, passes through a negative wire set conduit, and bifurcates at a negative bifurcation to the first rotational motor wire set and the second rotational motor wire set;
e) the first rotational motor wire set extending from the positive bifurcation and the negative bifurcation, passing through the main rotational bar, and terminating at the first rotational motor; and
f) the second rotational motor wire set extending from the positive bifurcation and the negative bifurcation, passing through the main rotational bar, and terminating at the second rotational motor.

18. The tactile stimulating device of claim 17 further comprising:
a) the main rotational motor housing having a main rotational shaft aperture;
b) the motor mounting plate having a motor mounting plate aperture;
c) the electrical contact plate having a contact plate aperture;
d) the male mounting assembly having a mounting assembly aperture;
e) the main rotational shaft having a knob aperture;
f) the main rotational shaft cooperating with the main rotational shaft aperture, the motor mounting plate aperture, the contact plate aperture, and the mounting assembly aperture; and
g) a knobbed attachment bolt cooperating with the knob aperture to secure the connection of the main rotational shaft in the main rotational shaft aperture, the motor mounting plate aperture, the contact plate aperture, and the mounting assembly aperture.

19. The tactile stimulating device of claim 18 further comprising:
a) the tactile stimulating device having a hard wire power cord to a ceiling light junction box.

20. The tactile stimulating device of claim 18 further comprising:
a) the tactile stimulating device having a power cord plug in connection to a power source.
